(19) United States
(12) Patent Application Publication
(10) Pub. No.: US 2020/0291340 A1
(43) Pub. Date: Sep. 17, 2020

(54) Lattice For Cell Culture

(71) Applicant: Sepragen Corporation, Union City, CA (US)

(72) Inventors: Andrew B. Burns, Claremont, CA (US); Vinit Saxena, Pleasanton, CA (US); Michael Ian Phillips, Claremont, CA (US)

(21) Appl. No.: 16/818,993

(22) Filed: Mar. 13, 2020

Related U.S. Application Data
(60) Provisional application No. 62/818,057, filed on Mar. 13, 2019.

(51) Int. Cl. C12M 1/12; C12M 1/00; C12N 5/0775

(52) U.S. Cl. CPC C12M 25/14; C12N 5/0663; C12M 29/00; C12M 25/10

(57) ABSTRACT

A lattice structure for culturing cells in a bioreactor is effective for culturing high density cells and maintaining cell type homogeneity. The lattice structure includes a plurality of channels forming a set of channels, each of the plurality of channels extending between a first channel pore surface and a second channel pore surface and each of the plurality of channels having a first channel pore and a second channel pore altogether forming a plurality of channel pores on each of the first channel pore surface and the second channel pore surface, wherein each of the channel pores has an area of between about 0.01 mm² to about 1 mm², and wherein the lattice structure is made of a biocompatible rigid material having a Young's modulus value of at least 0.5 GPa.

This application claim priority to U.S. Provisional Application No. 62/818,057 filed on Mar. 13, 2019, the entire contents of which are incorporated herein by reference.

### FIELD OF THE INVENTION

The field of the invention is a lattice structure for culturing cells, and most especially a lattice structure for culturing adherent cells in a bioreactor.

### BACKGROUND

The following description includes information that may be useful in understanding the present invention. It is not an admission that any of the information provided herein is prior art or relevant to the presently claimed invention, or that any publication specifically or implicitly referenced is prior art.

A robust method for scalable bioreactor cell culturing is ideally designed to render high density and often times high purity cell yields. In most conventional bioreactors, cells grow in the liquid nutrient medium which also serves as the means by which dissolved oxygen is transported to the cell surface. Typically, air (e.g., oxygen (O2) and carbon dioxide (CO2)) is dissolved in the medium by bubbling gas into it followed by stirring or rocking of the medium to aid in its diffusion to the cells. The oxygen which is known to have poor solubility in culture medium is rapidly depleted as the cell density rises. Oxygen depletion and poor mixing results in apoptosis of the cells, and an eventual “crash” or death of the culture. To prevent rapid depletion of oxygen in the culture, the bubbling, stirring, and/or rocking rates are all increased over time to thereby increase oxygen delivery. Unfortunately, these actions cause an increase in shear rates which fragile (e.g., mammalian) cells cannot handle. In perfusion systems, rates of media exchange are often increased to enhance oxygen transport. However, this renders an increase in media consumption, and increased cost of goods.

To reduce shear stress on more sensitive cells, cellulose-based matrices in a bioreactor system using gravity and capillary action have been used in which the cells are captured in the pores of the cellulose-based matrix. While cellulose-based matrices provide a low shear environment, for the culturing of adherent cells such as human mesenchymal stem cells (hMSCs), cellulose-based matrices are not effective for promoting adhesion of these anchorage-dependent cells some of which may also require high purity—e.g., maintaining potency stemness.

Thus, there remains a need for a robust method of culturing cells in a bioreactor that provides a low shear stress environment and yields high density and high purity/homogeneity of cells.

### SUMMARY OF THE INVENTION

The inventive subject matter provides an apparatus, systems, and methods in which a lattice structure has repeating channels throughout to allow for non-turbulent media flow through the lattice, while providing an effective amount of surface area for robust growth of adherent cells.

Aspects of the contemplated lattice structure for culturing cells include a plurality of channels forming a set of channels, each of the plurality of channels extending between a first channel pore surface and a second channel pore surface and each of the plurality of channels having a first channel pore and a second channel pore altogether forming a plurality of channel pores on each of the first channel pore surface and the second channel pore surface. Each of the channel pores has an area of between about 0.01 mm² to about 1 mm², and the lattice structure is made of a biocompatible rigid material having a Young's modulus value of at least 0.5 GPa.

In some embodiments, the lattice structure includes plurality of channels that are approximately uniform with no more than 2% variation between any two of the plurality of channels. Preferably, the first channel pore surface and the second channel pore surface have a diameter or diagonal of between about 5 mm to 100 mm. It is further contemplated that that the lattice structure has a total space-occupying volume of between about 2 cm³ to about 800 cm³.

Some embodiments include a lattice structure in which each of the channel pores is approximately the same size having no more than 2% variation in any dimension, and each of the channel pores has a diameter or diagonal of between about 0.1 mm to 1 mm. In similar embodiments, each of the channel pores of the lattice structure has approximately the same area size and the lattice structure further comprises a spacing distance between all of the channel pores that is equal to the area size of the channel pores.

In preferred embodiments, the lattice structure is made from a biocompatible rigid material having a Young's modulus value of at least 1.0 GPa. Exemplary biocompatible rigid materials include polylactic acid (PLA), polystyrene, polycarbonate (PC), polystyrene (PS), polyethylene terephthalate glycol (PETG), thermoset polyurethane (TPU), polycaprolactone (PCL), or acrylonitrile butadiene (ABS).

In some embodiments, the first channel pore surface and the second channel pore surface of the lattice structure has a perimeter edge forming a circular, oblong, square, octagonal, hexagonal, or rectangular shape. Additionally or alternatively, each of the plurality of the channel pores have a square or circular shape.

In additional embodiments, the lattice structure includes a hollow passage extending from the first channel pore surface to the second channel pore surface, the hollow passage allowing for insertion of a member for securing the lattice structure. In other additional embodiments, a removable substrate made of the biocompatible material and removably attached to at least one of the first channel pore surface or the second channel pore surface.

In more preferred embodiments, the lattice structure includes the first channel pore surface and the second channel pore surface each having a diameter or diagonal of between about 10 mm and 60 mm and each of the plurality of channel pores is of between about 0.2 mm and 0.4 mm, the lattice structure further comprising a spacing distance between each of the plurality of channel pores equal to the diameter or diagonal of the pores.

Additionally or alternatively, the lattice structure may have a surface coating. In particular embodiments, the surface coating includes a functional group capable of binding to the surface of a cell to be cultured. Additional surface coatings include denatured protein-based fibers, thermoresponsive polymers (i.e., thermopolymers), plasma treatment, and/or sodium hydroxide. Examples of denatured protein-based fibers include gelatin, collagen type 1, collagen type 2, fibronectin, and/or laminin. Examples of thermoresponsive polymers include poly(N-isopropylacrylamide) p(NIPAm), poly-(ethylpyrrolidone methacrylate) (pEPM), poly[2-(dimethylamino)ethyl methacrylate] (pDMAEMA), hydroxypropylcellulose, poly(vinylcaprolactame), and/or polyvinyl methyl ether.

A system for culturing cells is contemplated, wherein the system includes a bioreactor assembly including the presently disclosed lattice structure. In particular, the lattice structure may be used in/incorporated into a perfusion bioreactor for cell expansion and recovery. In some embodiments, the volume ratio of the lattice structure to the volume of the bioreactor is about 1:6 to about 1:8.

Additional aspects of the present disclosure include methods for maintaining cell type homogeneity. These methods include culturing a single cell type using the presently disclosed lattice structure at flow rates of about 0.25 ml/min to 0.5 ml/min. In preferred embodiments, methods of maintaining cell type homogeneity produce cell cultures having at least 95% cell type homogeneity.

Using the disclosed lattice structure incorporated into a bioreactor system allows for facile methods of seeding cells by gravitational or perfusion flow. In further embodiments, the culturing of cells on the lattice structure in a bioreactor system may be carried out under normal and hypoxic oxygen conditions. For example, the oxygen (02) levels may vary from 1% up to 20%.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

The patent or application file contains at least one drawing executed in color. Copies of this patent or patent application publication with color drawings will be provided by the Office upon request and payment of the necessary fee.

Various objects, features, aspects and advantages of the inventive subject matter will become more apparent from the following detailed description of preferred embodiments, along with the accompanying drawing figures in which like numerals represent like components.

FIG. 1A is a schematic of a bioreactor assembly 5 for cell culturing with a lattice structure 10 as presently disclosed, according to embodiments of the present disclosure.

FIG. 1B is a photograph of a bioreactor assembly 5 for cell culturing with a lattice structure 10 as presently disclosed, according to embodiments of the present disclosure.

FIG. 1C is a schematic showing a front-on view of a bioreactor assembly with a lattice structure 10 as presently disclosed with the lattice structure depicted to be suspended out of media and fluid is recirculated through the bioreactor system, according to embodiments of the present disclosure.

FIG. 2 shows a schematic (left) of a lattice structure according to some embodiments of the present disclosure where the lattice structure 10 has a first channel pore surface 20a and a second channel pore surface 20b. A photograph (right) of a lattice structure 10 with the plurality of channel pores 15, core passage 25, and sampling ledge members 30 depicted, according to some embodiments of the present disclosure.

FIG. 3A is a schematic showing sequential (left to right) layer three-dimensional (3D) printing of the lattice structure, according to embodiments of the present disclosure.

FIG. 3B is a schematic depicting one embodiment of forming the channels and channel pores of the lattice structure, according to embodiments of the present disclosure.

FIGS. 4A, 4B, 4C, 4D, 4E, and 4F are each scanning electron microscope (SEM) images of one or a few channel pores in a polylactic acid (PLA) 3D printed lattice structure where FIG. 4A is a view of an exterior side of the lattice structure. FIG. 4B is a view of an interior section of the lattice structure. FIG. 4C is a top-down view of the lattice structure. FIG. 4D is a zoomed in image of the exterior side of FIG. 4A. FIG. 4E is a zoomed in image of the interior section of FIG. 4B, and FIG. 4F is a zoomed in top-down view of FIG. 4C. The red squares denote zoom seen in second row.

FIG. 5 shows the results from Computational Fluid Dynamic (CFD) modeling on a modeled lattice structure where the upper model is a velocity contour showing the calculated results of fluid velocity in the lattice structure and the lower model shows the calculated results of shear stress in the lattice structure, according to embodiments of the present disclosure.

FIG. 6 is a graph of inlet flow rate versus average wall shear where the lattice structure was modeled in ANSYS and tested at various flow rates using Fluent. The strain rate was converted into shear stress using Equation 1, according to embodiments of the present disclosure.

FIG. 7 shows human mesenchymal stem cells (hMSCs) imaged on a PLA lattice structure used in a bioreactor. The hMSCs cells underwent a 3 day prime at 1.5% oxygen and were then cultured out for 7 day, and then stained with phalloidin (red) and DRAQ5 (blue). The two upper images show hMSC on a single fiber. The lower left image is a low magnification showing hMSC coverage among parallel fibers. The lower right image is projected Z-stack of fibers showing cell coverage. Center image shows top view (XY projection), and the top and side bars in this image show sideways projection (ZX and ZY).

FIGS. 8A-8D are data graphs from a hMSC bioreactor culture of varying oxygen tension compared to static hMSC culture grown under the same oxygen amounts and harvested on day 7. Static refers to t75 tissue culture flasks and dynamic refers to bioreactor culture. FIG. 8A shows the results of cell growth rate (i.e., doubling time) on the bioreactor and static hMSCs with respect to the oxygen tension. FIG. 8B is a graph of the fold increase in the culture growth rate for the hMSC bioreactor and static cultures of FIG. 8A. FIG. 8C is a graph of cells per cm². FIG. 8D is a graph of the specific growth rates and doubling times of hMSC cells grown on a lattice structure in a bioreactor according to embodiments of the present disclosure and hMSC cells grown in static culture.

FIG. 9 is an exemplary graph of data obtained from flow cytometry of hMSC grown on a lattice structure in a bioreactor system according to embodiments of the present disclosure in which hMSC cells were harvested day 7 from varying oxygen tension. CD105, CD73, CD19 and CD14 stained cells were analyzed using flow cytometry. No significant differences were found in marker expression after preconditioning using 0%, 1%, 1.5%. 5%, and normoxic gasses.

FIG. 10A is an exemplary graph of hMSC biomarker characterization using flow cytometry. The hMSC cells were cultured in both static and bioreactor (with lattice structure) and compared using CD105, C73, CD19, and CD14 staining, according to embodiments of the present disclosure.

FIG. 10b shows exemplary data of an overlaid flow cytometry image of 1.5% 02 primed hMSC cultures from day 7 in dynamic bioreactors (with lattice structure) showing positive markers and negative markers, according to embodiments of the present disclosure.

FIG. 10C shows exemplary data of an overlaid flow cytometry image of 1.5% 02 primed normoxic polystyrene static control culture showing positive markers and negative markers, according to embodiments of the present disclosure.

FIG. 11 shows exemplary light microscope images of stem cell induction in which cells were harvested day 7 from bioreactor (with lattice structure) and cultured in respective differentiation media following specialized protocols. The top row of images show osteocyte cell induction, the middle row shows adipocyte cell induction, and the bottom row shows chondrocyte cell induction. After the allotted time, cells were fixed, stained, and imaged using light microscopy. Scale bars are 100 microns. First column of images are stain controls of respective cultures.

FIGS. 12A and 12B show exemplary data comparing culture methods including dynamic bioreactor (with PLA lattice structure), spinner flasks, and static growth in t75 flasks as indicated, in which FIG. 12A presents doubling time and FIG. 12B presents the specific growth rates of the corresponding hMSCs. Static cultures were grown in t75 flasks according to ATCC guidelines. Spinner cultures used Cytodex-1 microcarriers in spinner flask. Dynamic culturing including use of a PLA lattice structure used PLA lattice as per method.

### DETAILED DESCRIPTION

The inventors have contemplated a lattice structure for culturing cells with high density yields while maintaining cell type homogeneity. The lattice structure is porous and made from a rigid biocompatible material. The porous and rigid lattice design provides for increased surface area and effective media and oxygen exposure under low shear conditions (e.g., low flow rate). Accordingly, the advantageous design enables robust growth of cells yielding high density cultures with high cell purity—i.e., cell type homogeneity.

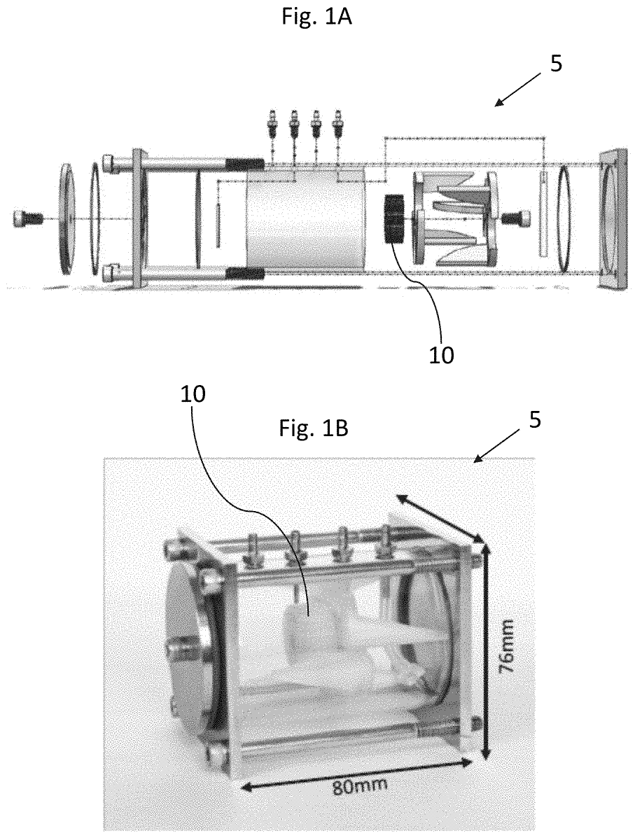

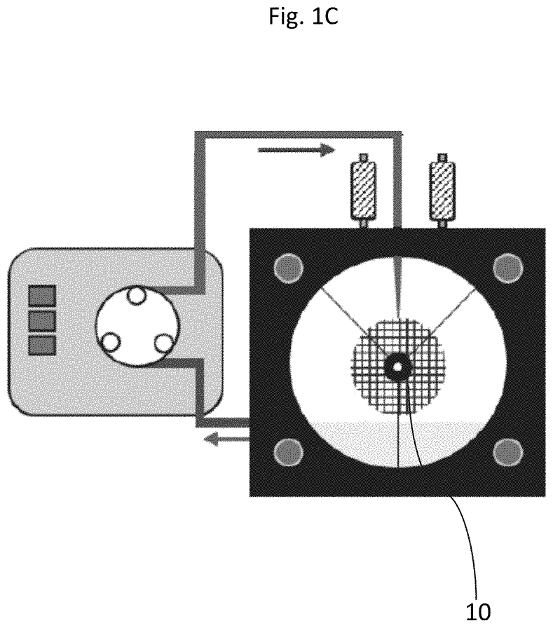

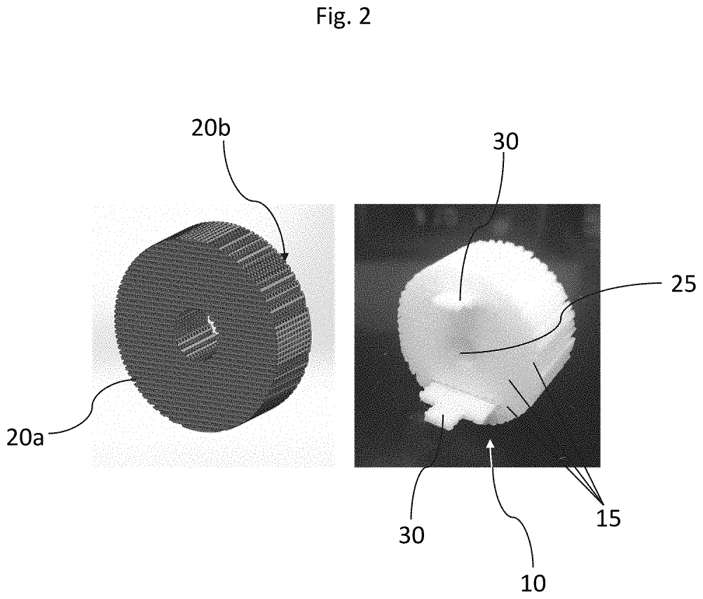

With reference to FIGS. 1A-1C, the contemplated features of the lattice structure 10 allows for its use in a bioreactor culturing assembly 5. More specifically, with reference to FIG. 2, the lattice structure 10 is made from a biocompatible rigid material and includes a plurality of channels 15 extending between a first channel pore surface 20a and a second channel pore surface 20b and each of the plurality of channels having a first channel pore and a second channel pore altogether forming a plurality of channel pores on each of the first channel pore surface and the second channel pore surface. Typically, each of the channel pores has an area of between about 0.01 mm² to about 1 mm², and the biocompatible rigid substrate material having a rigidity as measured by the Young's modulus value of the material, in which the rigidity is at least 0.5 gigapascals (GPa).

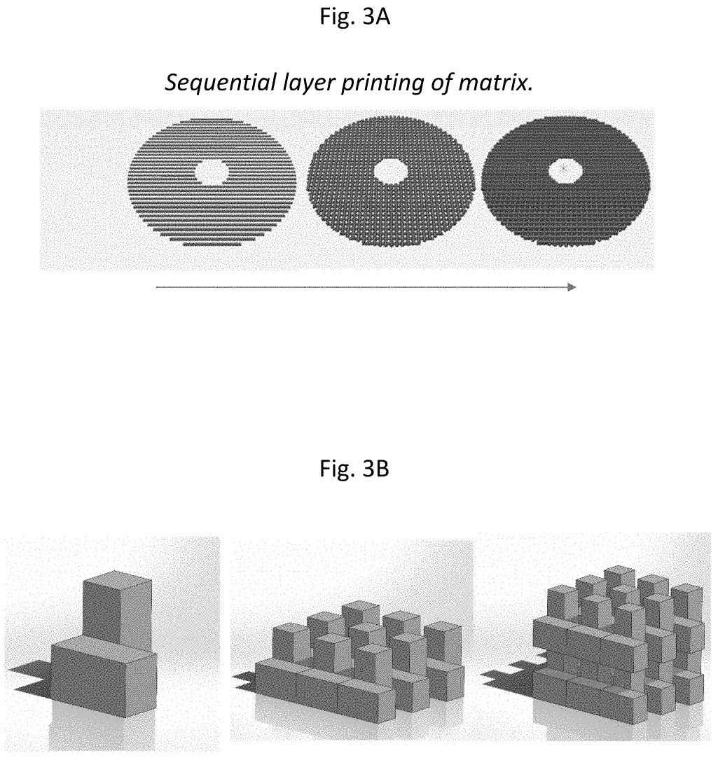

In some embodiments, the plurality of channels 15 in the lattice structure 10 are uniform in size and distribution throughout the structure to allow for even distribution of media received by gravity flow. Although any suitable manufacturing method and material may be used, the uniformity of the channels also allows for facile modular fabrication. Suitable manufacturing methods include casting or molding, as well as three-dimensional (3D) printing, computer numerical control (CNC) machining (e.g., CNC milling), selective laser sintering (SLS) (e.g., SLS 3D printing), injection molding, and photolithography. With reference to FIGS. 3A and 3B, fabrication of lattice structure is suitable for 3D printing in which the channels making up the lattice structure can be sequentially layered (FIGS. 3A-3B) allowing for facile fabrication and the advantageous uniformity and distribution of the channels and pores throughout the lattice structure. In an exemplary method using 3D printing, the lattice design is cut into many XY layers using computer software, known as slicing software. Then the printer type heats a material substrate to melting and extrudes the material though a nozzle, creating a very fine filament to complete a full layer. The extrusion head will then increase height in the Z axis and deposit another layer on top of the last. The heat of the filament causes the second layer to fuse to the first and the 3D object is slowly built up by subsequent layer addition.

While a lattice structure having non-uniform or less uniform channels is contemplated, a lattice structure having uniform channels is preferred. The uniformity provides even distribution of media and oxygen to the cells and allows for less turbulence surrounding the structure. Additionally, the manufacturing of a uniform lattice structure allows for easy modular fabrication. While uniformity can be readily achieved using any suitable method, approximate uniformity accounting for minor size variations is contemplated. Variations in channel and pore size and positioning may vary within the lattice structure. As such, uniformity as used herein also includes approximate uniformity and approximately uniform to include variations in any dimension of no more than 2%. Preferably, the variation in any dimension is no more than 1, 0.9, 0.8, 0.7, 0.6, 0.5, 0.4, 0.3, 0.2, or 0.1%.

The contemplated lattice structure may be used as cell culturing platform for any cell type. Any cell type includes animal (e.g., mammalian), yeast, bacterial, and plant cells. Notably, the lattice structure is conducive for growing adherent cells which require anchoring to a surface for growth. In addition to the lattice structure material being biocompatible, the material surface should also have some rigidity for effective adherence of the cells. The inventors contemplate a rigidity greater than that of cellulose and cellulose-based fibers. Typically, the rigidity of the biocompatible substrate material has a Young's modulus value of at least 0.5 gigapascals (GPa). More typically, the rigidity of the biocompatible substrate material is greater than 0.5 GPa. In preferred embodiments, the rigidity of the biocompatible material is at least 0.6, 0.7, 0.8, 0.9, 1.0, 1.1, 1.2, 1.3, 1.4, 1.5 GPa. More preferably, the rigidity of the biocompatible material is at least 1.0 GPa. For example, the rigidity of the biocompatible material may be of between 1.0 GPa to 4.5 GPa. Most preferably, the rigidity of the biocompatible material is of between 1.0 GPa to 4.0 GPa. For example, the rigidity of biocompatible material may be 1.0, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 GPa. Any suitably rigid biocompatible material may be used. Exemplary materials for fabrication of the lattice that are suitably rigid and biocompatible include polylactic acid (PLA), polycaprolactone (PCL), acrylonitrile butadiene styrene (ABS), polycarbonate (PC), polystyrene (PS), polyurethane (PU) (e.g, thermoset polyurethane (TPU)), and polyethylene terephthalate glycol (PETG). Selection of a rigid biocompatible material based on the requirements and restrictions for a particular bioreactor or cell culture assembly. Biocompatibility of a material includes sterilization, and any selected material can be sterilized by any suitable sterilization method. For example, heat sterilization at 121° C. (250° F.) or 132° C. (270° F.) (e.g., autoclaving) or by gas or gamma irradiation sterilization. The selected rigid biocompatible material would be selected to have the required rigidity as well as be able to withstand (e.g., without any notable or permanent change to the material) a suitable means of sterilization.

The contemplated lattice structure may be of any suitable size. While the lattice structure as disclosed herein may be used in a bioreactor assembly system, the use of the lattice structure is not limited to a bioreactor assembly system. Accordingly, the lattice structure as disclosed herein may be fabricated to have any size and dimension that is limited only by the capability to manufacture the lattice structure. As specifically exemplified herein, a lattice structure for use in a bioreactor assembly system was fabricated to a size that can be incorporated into the bioreactor assembly system. The skilled person could readily follow this present disclosure to increase the size of the lattice structure, as the disclosed methods and protocols for fabricating a lattice structure of the presently disclosed size and dimensions are applicable for larger as well as smaller structures and would merely be restricted by the limitations of the manufacturing process. For example, the presently disclosed lattice structure may be fabricated by 3D printing. While the methods disclosed herein may be easily calculated to produce a lattice structure on a much larger scale (e.g., 6 inches, 1 foot, or 3 feet in size for at least one dimension), such fabrication may be limited, for example, by access to or the existence of a 3D printer capable of producing a structure of that size. As such, while 3D printing may be a more straightforward and facile manufacturing method, any suitable method including the more laborious casting or molding, as well as CNC machining (e.g., milling), SLS (e.g., SLS 3D printing), injection molding, or photolithography may be used for forming a lattice structure from a rigid biocompatible substrate.

While any size of the lattice structure is contemplated, for use in most bioreactor culturing systems, the lattice structure is sized accordingly. Typically, the lattice structure has a total space-occupying volume of between about 2 cm³ to about 800 cm³. For example, the lattice structure may have a volume of between about 2 cm³ to about 400, 500, 600, 700, or 800 cm³. More typically, the lattice structure has a volume of between about 10 cm³ to about 100, 150, 200, 250, 300, 350, or 400 cm³. Most typically, the lattice structure has a volume of between about 20 cm³ to about 40, 50, 60, 70, 80, 90, or 100 cm³.

The increased surface area provided by the contemplated lattice structure enables high density growth of cells, especially adherent cells. This increased surface area is provided by the channels formed throughout the lattice. With reference again to FIG. 2, at the ends of each channel are the pore openings 15 which altogether form a channel pore surface 20a, 20b on each side of the lattice 10. Typically, these channel pore surfaces have a diameter or diagonal of between about 5 mm up to 100 mm. More typically, the diameter or diagonal of the channel pore surface is of between about 10 mm to 40, 50, 60, 70, 80, 90, or 100 mm. Most typically, the diameter or diagonal of the channel pore surface is of between about 20 mm to 60 mm. For example, the diameter or diagonal of the channel pore surface may be 20, 25, 30, 35, 40, 45, 50, 55, or 60 mm.

The layout of the channels in the lattice structure is not limited. For facile and modular fabrication, the channels are contemplated to be formed equidistant apart in parallel rows as shown in FIG. 2. Typically, each of the channel pores are approximately the same size having no more than 2% variation in any dimension. In particular, for increased uniformity and fabrication, the lattice structure may have a distance that is the same for the width and length of the pores as well as the space in each direction between all of the pores, such that the area of the pore is equal to the area of the space between the pores for at least 70% of the channel pore surface. More typically, the area of the pores and the space between the pores is uniform (with no more than 2% variation) for at least 80, 85, or 90% of the channel pore surface.

The overall shape of the lattice structure is not necessarily limited. Exemplary shapes of the channel pore surface that may be more readily manufactured include a circle, an oblong, a square, an octagon, a hexagon, or a rectangle. More preferably, the channel pore surface has a circular or square shape. Additionally, the shape of channels and pores are not limited, but are more easily fabricated in a square or circle having a diameter or diagonal of between about 0.2 mm and 0.4 mm.

Notably, the presently disclosed lattice structure allows for robust cell culturing of cells. In particular, for adherent mammalian cells (e.g., human MSCs), because the lattice structure allows for sufficient surface area for growth and sufficient wetting. Furthermore, the lattice channels allow for low flow rates of media surrounding the lattice structure—e.g., between 0.25 to 0.50 ml/min. As such, more sensitive cells like hMSCs can grow robustly and without turbulence/shear stress that can also induce differentiation.

In particular, as further detailed herein, using Computational Fluid Dynamic (CFD) modeling, very low velocities and subsequently low shear occur inside the lattice structure. Specifically, the computational modeling of this lattice structure reports maximum values of 0.0054 dynes cm⁻². By comparison, CFD of stirred tank reactors utilizing microcarriers report values of approximately 1 to 5 dynes cm⁻². See, Table 1, below. Tubular systems with similar laminar flow patterns report average values of 0.98 dynes cm⁻². These values all fall within 0.02 to 9 dynes cm⁻², a range shown to upregulate osteogenic genes and differentiation in hMSCs. However, the presently disclosed lattice structure was determined to have two orders of magnitude lower than this reference range.

TABLE 1

| | | | | | | | | | | Total | | Shear |
|---|---|---|---|---|---|---|---|---|---|---|---|---|
| | | | | V | SA | | Cell | SC/mL | SC/cm² | SCs | Td | (dynes |
| Name | Type | Classification | Vendor | (mL) | (cm²) | BA:V | type | x10³ | x10⁴ | x10³ | (hr⁻¹) | cm⁻²) |
| — | POMS | Immobilized | — | 110 | 2,800 | 26.5 | hP- | 0.509 | 2.00 | 56 | 30.2 | 8-5 |
| | Matrix | | | | | | MSC | | | | | |
| Quantum | Hollow | Immobilized | TERUMO | 1,440 | 21,000 | 14.6 | hAd- | 0.167 | 1.14 | 240 | 34.1 | 0.3-0.7 |
| | Fiber | | BCT | | | | MSC | | | | | |
| Mobius | STBR | Suspension | M8ipore | 50,000 | 800,000 | 6 | hBM- | 2.00 | 1.68 | 5,000 | 54.0 | 2-40 |
| | | | Sigma | | | | MSC | | | | | |
| Applmex | Wave | Suspension | Applikon | 1,500 | 7,360 | 4.91 | hAd | 0.190 | 3.87 | 285 | 31.2 | 0.1-0.5 |
| | Bug | | | | | | MSC | | | | | |
| Mag 3 | Paddle | Suspension | PBS | 3,000 | — | — | hBM- | 1.90 | — | 5,700 | 63.0 | — |
| | | | | | | | MSC | | | | | |
| Xpansion | Parallel | Immobilized | Pa8 | 1600 | 6,120 | 3.83 | hAd- | 0.181 | 5.4 | 334 | 34.1 | 0.1 |
| Multiplate | Plate | | | | | | MSC | | | | | |
| ICel8s | Random | Immobilized | Pa8 | 1000-5000 | 40,000 | 40 | hBM- | 2.93 | 16 | — | 67.2 | 1-5 |
| | Fiber | | | | | | MSC | | | | | |
| | Matrix | | | | | | | | | | | |
| In House | Lattice | Immobilized | — | 20 | 122 | 6.3 | hBM- | 0.2 | 2 | 1.8 | 82 | 0.0042 |
| | | | | | | | MSC | | | | | |

Abbreviations:
Volume (V),
SA Surface Area (SA),
Stem Cels (SC),
Doubling Time (Td).

Advantageously, culturing cells on the rigid biocompatible surface of the lattice structure allows for improved and more efficient downstream harvesting and purification compared to other known methods, including culturing with microcarriers (e.g., beads). Microcarrier-based cell culturing of stem cells requires extra steps in order to purify the cells from the beads. For example, some beads require cell detachment using an enzyme (e.g., trypsin), resulting in an extra step of straining the microcarriers from the lifted cells. Furthermore, the byproducts of digestion of these microcarriers is still a concern for final formulation and patient administration. Pursuant to the United States Pharmacopeia (USP) <788> requirements, microcarriers as particulate matter should be removed from injected products. Thus, systems using microcarriers for hMSC therapies require either initial steps or straining and filtration steps to remove microcarriers from cells after dissociation, adding complication and potentially decreasing overall yield through shear. However, cells grown on the presently disclosed lattice structure can be washed in place and lifted with fewer processing steps and without the use of a harsh enzyme like porcine trypsin. For example, cells grown on the presently disclosed lattice structure may be dissociated from the lattice surface using Cell Dissociation Buffer (CDB) (Gibco) and/or a gentle enzyme solution (e.g., TrypLE™, Gibco). Furthermore, it is noted that a lattice structure made with polylactic acid (PLA) is further advantageous because the degradation product of PLA dissolves into solution as lactic acid which can be easily removed through buffer exchange, but it is also biocompatible and broken down in the body naturally.

In additional embodiments, the lattice structure includes a surface coating to enhance cell adhesion of any type of adherent cell. The surface coating may be applied to all or at least some of the available surface area of the lattice structure. In typical embodiments, the surface coating includes at least an application of functional groups to promote cell adhesion. More typically the surface coating includes denatured protein-based fibers, thermoresponsive polymers (i.e., thermopolymers), sodium hydroxide, and/or a plasma treatment.

The selection of a coating surface may depend on the type of cells to be cultured and their degree of sensitivity to the processes required to later detach the cells from the surface coating or treatment. Denatured protein-based fibers include gelatin, collagen type 1, collagen type 2, fibronectin, and/or laminin. For example, denatured collagen adheres to the lattice biocompatible material (e.g., PLA) and also provides a binding motif for the cells, thereby providing a sort of bridge between the lattice surface and the cells. Plasma treatment of the lattice surface exposes hydroxyl groups in the surface material, thereby provide a binding motif for cells. Additionally, thermoresponsive polymers may also be coupled to the surface of the lattice structure. Examples of these temperature responsive polymers include poly(N-isopropylacrylamide) p(NIPAm), poly-(ethylpyrrolidone methacrylate) (pEPM), poly[2-(dimethylamino)ethyl methacrylate] (pDMAEMA), hydroxypropylcellulose, poly(vinylcaprolactame), and polyvinyl methyl ether. Preferably, the thermoresponsive polymer is p(NIPAm) or pEPM. Accordingly, a thermoresponsive polymer may be coated onto the lattice structure followed by the culturing of cells which attach to the polymers on the lattice surface. With a change in temperature, the cells detach from the polymers. See, e.g., Nash et al., 2012, J. Mater. Chem. 22, 19376, the entire content of which is incorporate herein by reference. These cell dissociation processes are less complicated than conventional protocols and are less destructive to the cells, thereby allowing for higher cell yields without the removal of undesirable components (e.g., trypsin or microcarrier beads).

Considered from a different perspective, because of its biocompatibility, cell detachment from the lattice structure, may not be necessary depending on the desired application. For example, as PLA is biocompatible and similar in rigidity to cancellous bone, hMSCs may be expanded and differentiated in-situ. In this way, polymer rigidity may be either avoided or exploited for tailored stem cell differentiation. Harder polymers like PLA, polystyrene, or polycarbonate (PC) may be printed using high temperature 3D printers, and can be easily treated for cell adhesion. Softer, more elastic materials like polyurethane (PU) have been used for stem cell culture and are also readily available materials for 3D printing. Furthermore, culture on more elastic scaffolds, such as alginate encapsulation, may direct hMSCs to differentiate into chondrocytes and has been used in established differentiation protocols. See, e.g., Elsawy et al., 2017, Renew. Sustain. Energy Rev. 79, 1346-1352 and Fromstein et al., 2008, Tissue Eng. Part A, 14, 369-378, the entire contents of both of which are incorporated herein by reference.

As such high density “pure” (homogenous) cells can be effectively cultured using the presently disclosed lattice structure. Methods for maintaining cell type homogeneity (e.g., cell pureness or stemness), include seeding cells in a bioreactor culture system using the lattice structure disclosed herein. As detailed herein, cell type homogeneity may be maintained up to at least 95% up to at least 9 culture passages. Preferably, cell type homogeneity is maintained up to least 97% through at least 9 culture passages. Typically, cell type homogeneity is maintained up to at least 95% through 6 to 9 culture passages. More typically, cell type homogeneity is maintained up to at least 97% through 6 to 9 culture passages. As disclosed in further detail herein, hMSCs grown in a bioreactor using a lattice structure as disclosed herein, produced higher cell density yields compared to other systems, while maintaining cell type homogeneity as measured by expression of stem cell biomarkers. Furthermore, the cells lifted from the lattice structure surface were easily dissociated with open-air steps and required no extra purification. When tested for stemness (i.e., cell potency), the cells readily differentiated into osteocytes and were able to differentiate into adipocytes.

The contemplated lattice structure may also be utilized for the production of secreted product, as cells are adherent to a stationary scaffold. The disclosed lattice structure is ideal for secreted proteins and vesicles. The cells are bound to the lattice structure surface and will release cytokines and exosomes of therapeutic interest into circulating media. Studies of exosomes has shown their usefulness in wound healing and inflammatory diseases. These vesicles are secreted by hMSCs and contain mRNA, cytokines, growth factors, and other signaling molecules involved in healing, and are a major interest for regenerative medicine. The presently disclosed lattice structure may be run in perfusion, allowing simple harvest of the secretome while cells are held stationary in the bioreactor.

The inventors have further contemplated a lattice structure having a removable sampling shelf 25 (FIG. 2) to be attached to or formed on one or both of the channel pore surfaces. The removable sampling shelf allows for a sample of the cell culture to be removed for observation—e.g., to assess culture confluency and health of the cells. The removable sample shelf may be made of any biocompatible rigid material and is preferably made from the same biocompatible rigid material as the lattice structure.

Additionally, the inventors further contemplated a lattice structure adapted to be placed securely in a bioreactor vessel. With reference to FIG. 2, a hollow passage 30 extends between the channel pores surfaces and allows for insertion of a support member through the lattice structure with minimal disruption to the media flow around the lattice structure and the surface area for cell growth.

The following Examples are presented for illustrative purposes only, and do not limit the scope or content of the present application.

### EXAMPLES

### Example 1

A Scaled Bioreactor Culture System. From 3D modeling in Solidworks it was calculated that a 30 mm diameter lattice structure (e.g., as shown in FIG. 2) as disclosed herein, has a theoretical surface area of 225 cm². As a comparison, each 30 mm diameter repeating layer provides 23.5 cm², which equates to a 32-fold increase in surface area when comparing the 3D lattice to the equivalent 2D culture area.

### Example 2

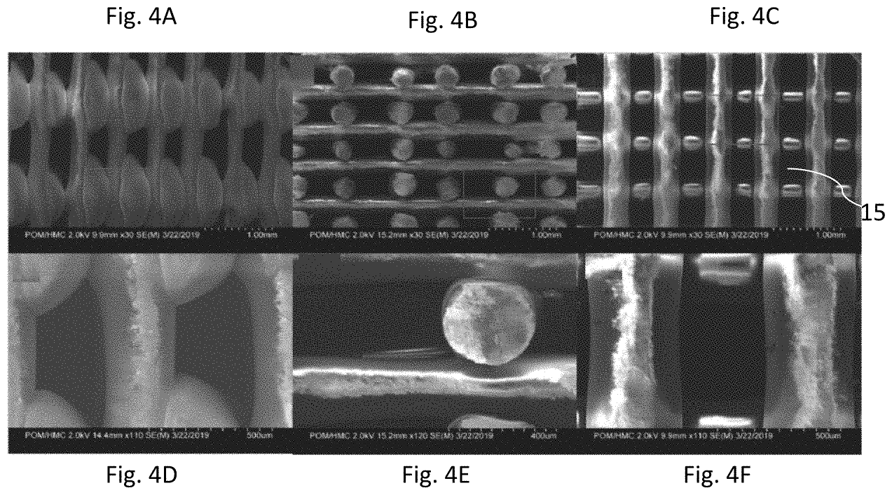

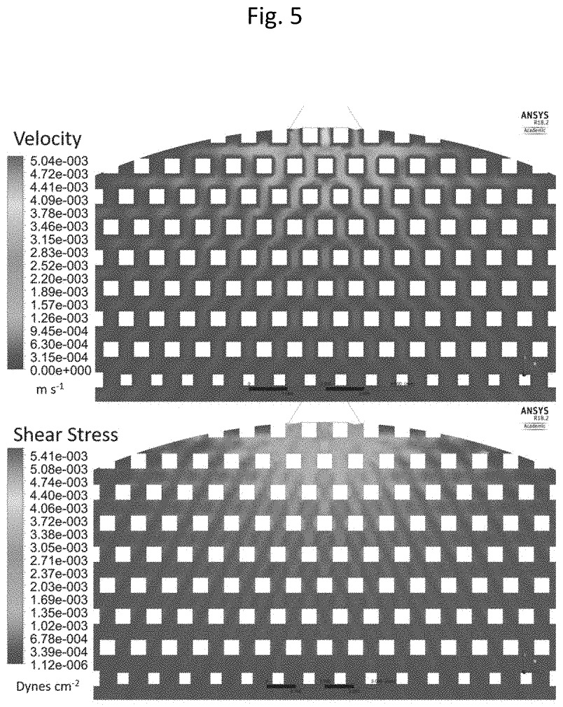

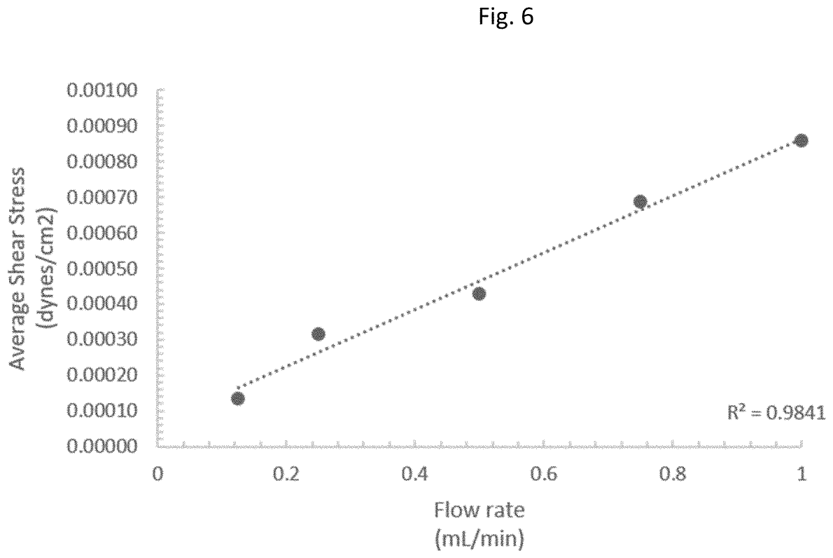

CFD Modeling. To demonstrate the principle of a reactor using the disclosed lattice structure and extrapolate hydrodynamic forces in the lattice, ANSYS FLUENT was used with a simplified model of the growth lattice. SEM images (FIGS. 4A-4F) of the scaffold were taken to understand the printed geometries and properly model the system in FLUENT (FIG. 5). A dye tracer benchtop experiment was used to validate the model. As mentioned, media is cycled to the top of the circular lattice and pulled by gravity through the pores. Because mixing is accomplished through passive means rather than an impeller, the system is inherently very low shear. This was proven by testing a range of flow rates to estimate shear vs flow rate (FIG. 6). All prospective flow rates fell well below 0.4 dynes cm⁻². As 0.25 mL min⁻¹ resulted in the lowest shear while keeping the matrix well wetted, it was the tested flow rate for hMSC culture. At this flow rate CFD modeling reported a maximum of 0.0059 dynes cm⁻² (FIG. 5) and an average of 0.0031 dynes cm⁻² (FIG. 6). The areas of highest shear were at the top and bottom center of the matrix insert, where the media was entering and exiting the lattice respectively.

### Example 3

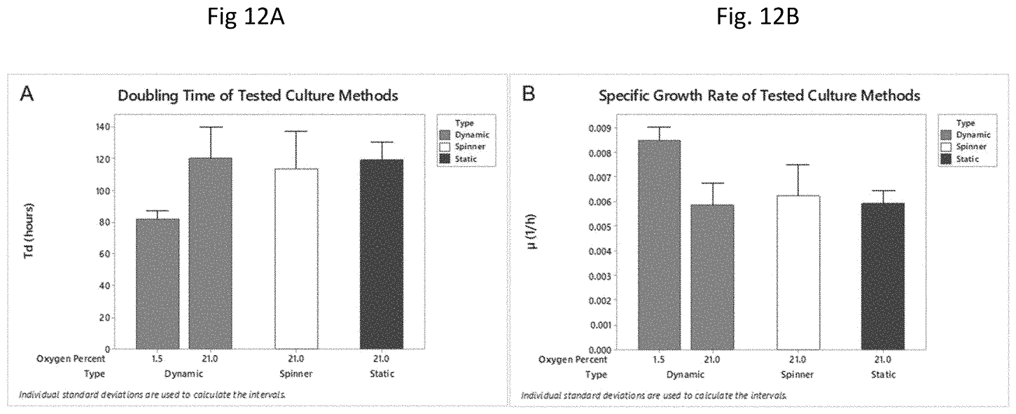

Spinner flask control. As a comparison, hMSCs growth was also investigated on Cytodex-1 microcarriers in small scale spinner flasks. Static cultures (n=6) showed an average doubling time and specific growth rate of 119.07 hrs±11.23 and 0.0062 hr⁻¹±0.0013 respectively (FIG. 12A). Cells cultured in spinner flasks showed an average doubling time of 113.6 hrs±23.75 and a specific growth rate of 0.0062 hr⁻¹±0.0013 (n=3) (FIG. 12B). Both are significantly longer (p=0.002) compared to lattice reactor (n=5) results.

### Example 4

Cell viability on polylactic acid (PLA) Lattice. Cell viability was compared between culture substrates and static vs dynamic cultures as previous studies with fibrous matrices exhibited increased cytotoxicity. On day 7 of cultures, cells were enzymatically lifted and viability was tested via trypan blue staining. PLA lattices were removed from culture wells to isolate only cells adherent to the PLA lattice. Dynamic PLA cultures from the bioreactor had an average viability of 96.54%±2.82. Cells grown in dynamic bioreactor culture on PLA showed no statistically significant difference (p=0.98) from static PLA culture plates, with an average viability of 96.76%±3.84. Dynamic PLA showed no difference (p=0.45) from Static PS, which had an average viability of 95.13%±1.07. This is also in agreement with the fact that static PLA and PS showed no statistical difference in viability (p=0.38). Therefore, PLA showed no detrimental effects on cell viability in both static and dynamic cultures compared to conventional culture on treated polystyrene flasks.

### Example 5

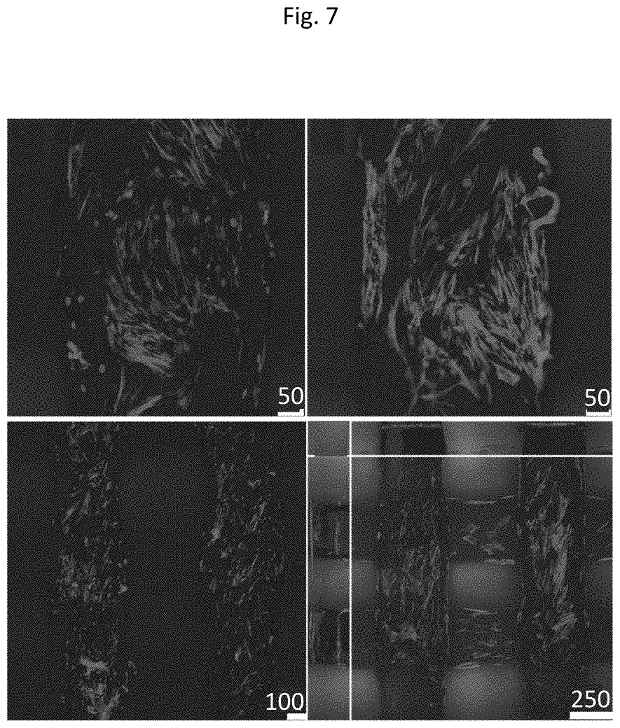

Dynamic seeding. Because of the larger channel sized and homogeneity of the lattice, a new seeding protocol was developed to increase seeding efficiency. The method that yielded the best results was through static settling of the cells. The volume of media the lattice could hold was found to be 2 mL. Thus, 500,000 cells were resuspended in 2 mL of hMSC media. This cell rich media was then slowly injected through a Luer lock until liquid had cleared the lines. The reactor was then placed into the incubator for 45 minutes to allow cells to settle onto lattice and adhere. Hypoxic gas was then overlaid into the system though the filter ports and the peristaltic pump was then started. After 7 days cells formed confluent monolayers towards the top center of the lattice sampling shelf (FIG. 7).

### Example 6

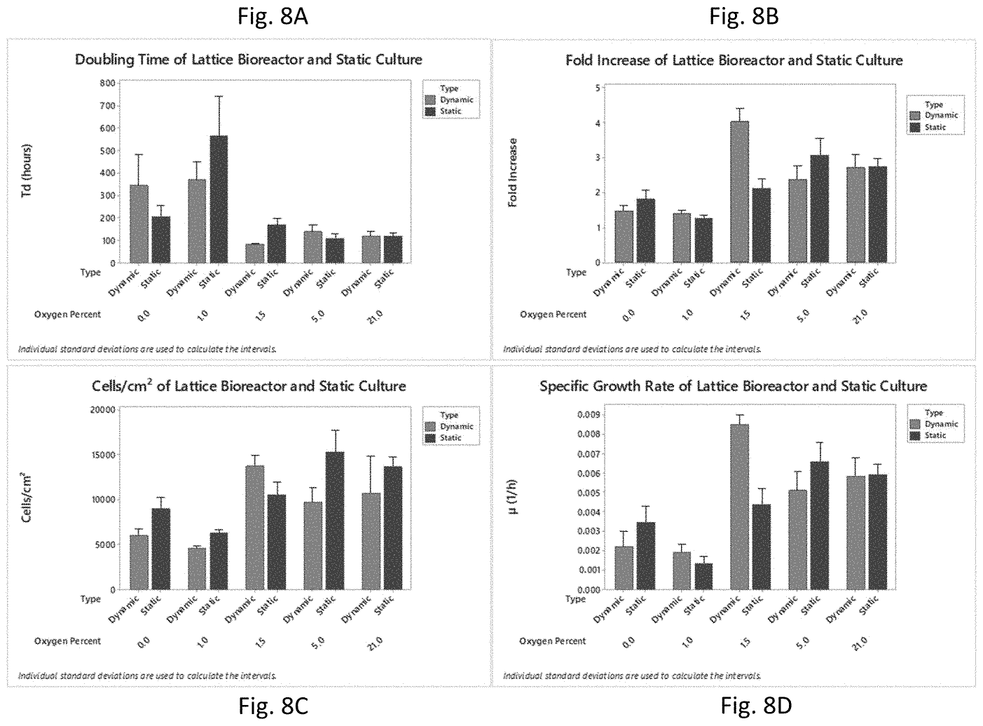

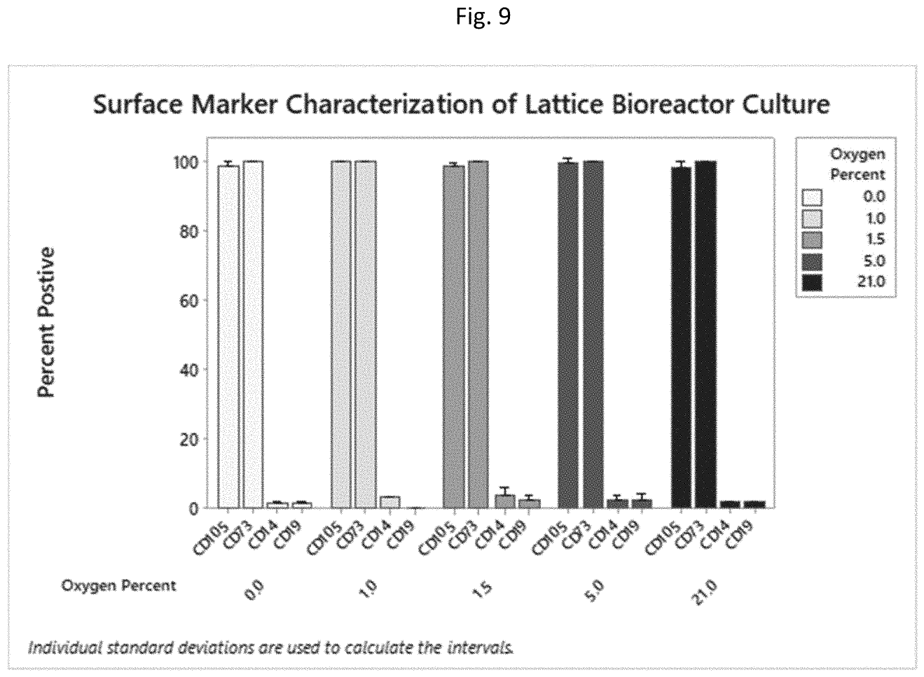

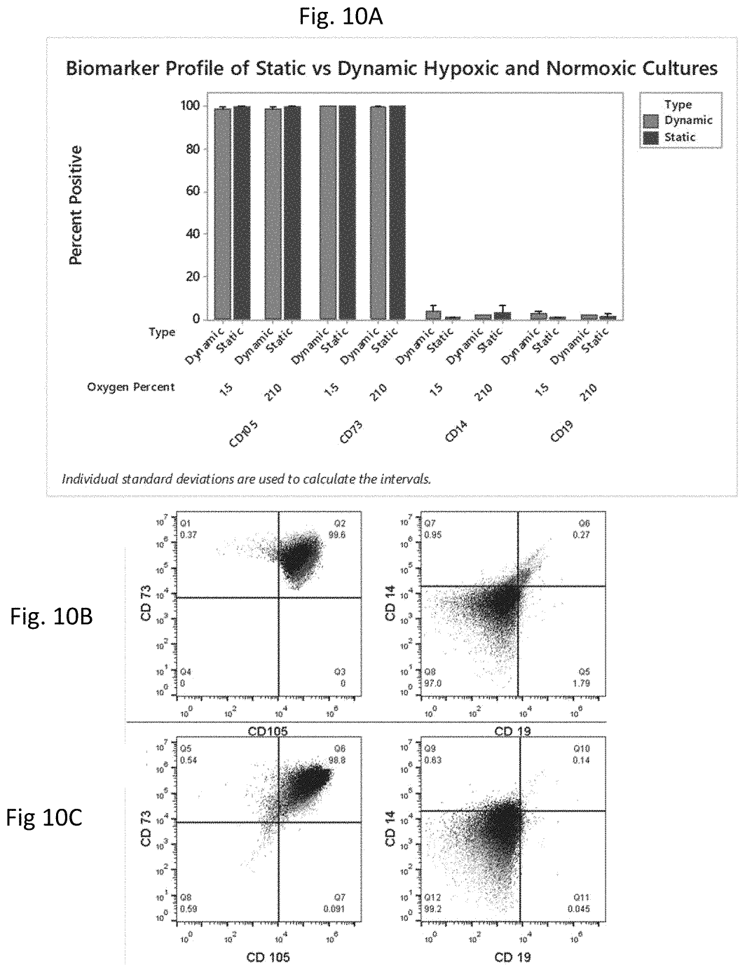

Reactor Culture. Normoxic reactor culture resulted very similar doubling time as polystyrene (PS) control cultures (FIG. 8A). Because hMSCs normally grow in more comparatively more hypoxic conditions in vivo, oxygen tension was investigated as a means of increasing cell proliferation. It was found that 1.5% O₂ resulted in a four-fold increase in cell yield; double that of conventional flask culture methods tested (p<0.001) (FIG. 8B). Normalized yield to surface area was 13,725 cells cm⁻² at 1.5% O₂ (FIG. 8C). This in-situ conditioning resulted in the significant increase in specific growth rate (0.0085 h⁻¹±0.0005) (FIG. 8D). When lifted and analyzed via flow cytometry it was found that cells cultured in the bioreactor retained their biomarker phenotype regardless of gas composition used for hypoxic treatment (CD105+CD73+CD14− CD19−); ANOVA showed no significant difference in CD105 (p=0.309), CD73 (p=0.347), CD19 (p=0.676), and CD14 (p=0.523) biomarker expression (FIG. 9). Thus, oxygen tension had a drastic effect on cell proliferation, and no effect on biomarker profile. Cultures primed at 0% and 1% (n=3 for both conditions) produced statistically similar cell yields, and cultures primed at 5% and 21% oxygen showed no statistically significant difference via Tukey test at 95% CI. Compared to control cultures on static tissue treated PS, the dynamic bioreactor culture on PLA produced a higher purity MSCs according to ISCT standards, Lifted cells were over 98% dual CD105 and CD73 positive cells in reactor culture compared to 94% in static normoxic polystyrene culture (p=0.005) (FIG. 10A). There was no significant difference in the negative markers CD14 and CD19 under normoxic (n=9) or 1.5% hypoxic conditioning (n=6) (FIG. 10A), and single populations of cells were harvested from bioreactors (FIG. 10B, 10C). Again, cells formed monolayers on the PLA filaments much like control cultures on PS dishes.

### Example 7

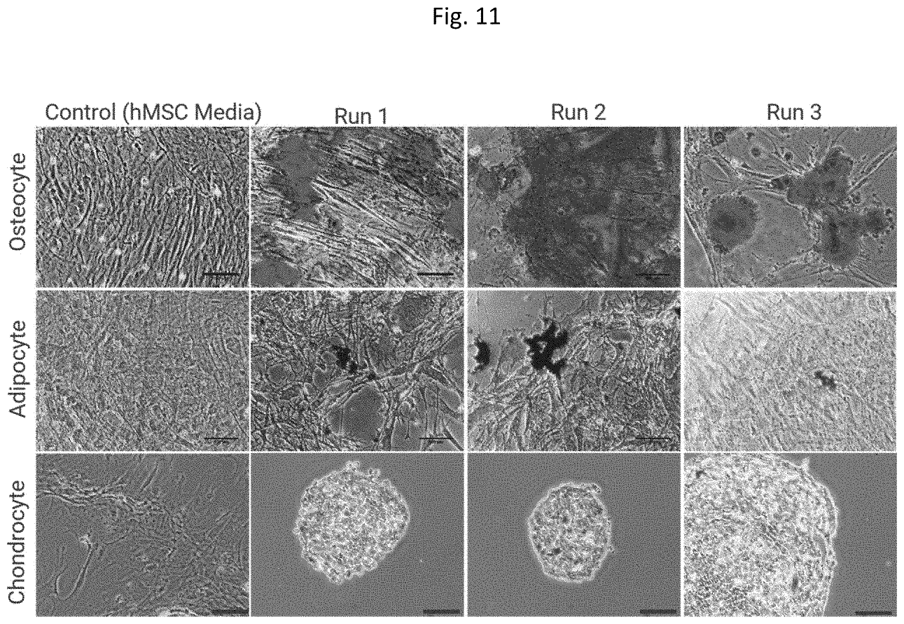

Differentiation potential. To test differentiation potential per ISCT guidelines, osteocyte, adipocyte, and chondrocyte inductions were performed stem cells harvested from 7 day bioreactor culture. For inductions, cells were cultured between 15 to 20 days in their respective, defined ATCC differentiation media, after which cells were washed, fixed and stained. After 7 days in bioreactor culture and hypoxic conditioning the cells retained their ability to differentiate into Adipocytes, Chondrocytes, and Osteocytes (FIG. 11). Control cultures were also done in parallel with the inductions and stained with the same dyes. Control cultures showed no staining in uninduced hMSC controls cultured for 21 days in hMSC media.

### Example 8

Stem cell culture. hMSCs were cultured according to guidelines provided from American Type Culture Collection (ATCC). Briefly, cells were cultured in hMSC media (ATCC PCS-500-030) supplemented with the bone marrow derived hMSC bullet kit (ATCC PCS-500-041) at 37° C. and 5% CO₂ on T-75 treated tissue culture flasks. A ¾ media exchange was performed on day 3, and cells were passaged at 80% confluency, usually on days 6 or 7. Cells were lifted using 3.5 mL of 0.25% trypsin and 0.53 mM EDTA solution (ATCC 30-2101) for regular passaging of T-75 flasks, and cells were re-plated at 5,000 cells cm⁻². Cell pelleting was performed by centrifugation at 270×g for 5 minutes. Working cell bank was created from passage 4 hMSCs and stored in liquid nitrogen. Experiments using hMSCs were conducted on cells between passage 5 and 9. Specific growth rate and doubling time were calculated to compare culture success.

$${T}_{d} = ( {T}_{2} - {T}_{1} ) * \frac{ln ( 2 )}{ln ( \frac{{q}_{2}}{{q}_{1}} )} \qquad Equation 1$$

Equation 1: Doubling time. Where T_{d} is the doubling time, q₂ is the final cell count, and q₁ is the initial cell seeding quantity.

$$μ = \frac{ln ( \frac{{q}_{2}}{{q}_{1}} )}{t} \qquad Equation 2$$

Equation 2: Specific growth rate. Where μ is the specific growth rate, q₂ is the final cell yield and q₁ is the initial cell seeding quantity, and t is the time between q₂ and q₁.

### Example 9

Oxygen tension studies. To induce low oxygen states, cells were placed in a hypoxia chamber (billups-rothenberg) and gas flushed for 6 minutes with the regulator set at 5 PSI and 10 L min⁻¹. Gas composition varied, but was mixed based on PSI. For reactor cultures the mixed gasses were introduced at 100 mL min⁻¹ for 5 minutes to exchange the head space and oxygen from the media. At first tri-gas mixture including 5% CO2 was used, but hMSCs preferred basic conditions and as such CO₂ was excluded from later bioreactor runs with negligible impact on yield and purity. For both cultures the hypoxic gas was overlaid for 3 days, at which point normoxic gas was reintroduced to the culture. For 0% O₂ treatment period had to be reduced to only 1 day.

### Example 10

Reactor construction. The chamber of the reactor is made of a 9 cm long polycarbonate tube with an ID of 2.25 in and an OD of 2.50 in. Four 316 stainless steel barbed hose adapters are tapped into the top of the polycarbonate, two for media circulation and two for gas exchange through 0.2 μm filters. Size 14 silicone hose was used for main liquid handing loop, with a 14 gauge Tygon Pharmed section for peristaltic pumping. The headplate and backplate are made of 316 Stainless steel. The interior reactor components include the 3D printed matrix, which is suspended out of the media using two brackets. Media pumped to the top of the lattice is perfused through the lattice design via gravity, providing gas exchange and nutrients to the cells. Gas control is highly tunable, as there is less liquid for gas to diffuse through to be available to the cells. The front plate of steel has a pass-through port for access to removable sampling scaffolds to monitor cell confluency. Both the lattice matrix and the holding parts were printed from PLA. Minimum and maximum working volumes used were 20 and 30 mL.

### Example 11

Lattice design and bioreactor culture. PLA matrices were 3D printed using a PrintrBot Simple printer and Cura 3D (V3.2.1) printing software. A 0.4 mm nozzle diameter was used to print the PLA scaffold. The lattice is constructed such that the smallest features are printable with a conventional 3D printer, and allow ample space for cells to culture into monolayers. For this extruder the lower limit of resolution was 400 microns in the XY plane. The lumen between fibers was made to be the same width as the fiber itself. Also included into the design are two inserts for non-destructive means of visualization of cell confluence and viability via calcein staining. To sterilize parts before culture, the matrix and supports are assembled and placed into reactor and steam sterilized at 121° C. for 15 minutes under a dry cycle. After sterilization the matrix is washed and wetted with filtered and autoclave-sterilized lx PBS (VWR VE404) and gelatinized with filtered and autoclave-sterilized 0.1% (W/V) gelatin (Fisher 9000-70-8) in MQ water for 45 minutes at 37° C., or overnight at 4° C. The matrix is then rinsed with PBS to remove excess gelatin, and cells are seeded at 2,500 cells cm⁻². Approximate surface area was calculated using Solidworks (Waltham, Mass.) analysis function. To allow cells to adhere only in the lattice the desired number of cells were resuspended first in a total of 2 mL, as this volume was found to be the holding volume of the matrix. Cells were allowed to settle in the matrix for 45 minutes before starting the recirculation loop. Recirculation was run between 0.25 mL and 0.5 mL min⁻¹. A range is noted because as the peristaltic tube relaxed during use, the peristaltic pump tended to speed up, slightly increasing the overall rate. This was the allowable flow rate range because it was the slowest rate that still allowed complete matrix wetting. A ¾ media exchange was performed on day 3, and cells were harvested on day 7 using a lifting cocktail comprised of a 2:1 mixture of Cell Dissociation Buffer (CDB) (Gibco 13151014) and TrypLE-Express (Gibco 12604021). Lifting was accomplished by aspirating media out and cycling 10 mL of PBS through system at 1 ml min⁻¹ to remove residual media. PBS was then aspirated, and cell-lifting cocktail was added. The reactor was then cycled at 0.25 ml min⁻¹ for 30 minutes, or later at 1.5 mL min⁻¹ for 15 minutes. Viability and cell counting was performed using hemocytometer and trypan blue staining.

### Example 12

Microcarrier culture in spinner flask. Cytodex-1 microcarriers were weighed and autoclaved at 121° C. for 15 minutes. Microcarriers were then hydrated in hMSC media. Cells were seeded at 5,000 cells cm⁻² in 50 mL of media in a 250 mL spinner flask (Wheaton). For the first 24 hours, the spinner flask was set to 15 RPM to allow hMSCs time to adhere to the microcarriers, after which agitation was increased to 30 RPM and volume increased to 80 mL. A ½ media change was performed on day 3. Samples were drawn each day and fixed in 4% paraformaldehyde (PFA) for 15 minutes. Cells were then prepared for cell counting via DRAQ5 (Abcam ab108410), staining in a 5 mMol solution overnight. Samples were then washed twice with PBS, allowing microcarriers to gravity settle between washes. Samples were imaged on Leica SP5. The culture was run for a total of 7 days. On day 7 media containing microcarriers was split into 50 falcon tubes and microcarriers allowed to settle for 20 minutes. Media was aspirated and microcarriers washed twice with PBS. When settled again, TryplE was added and mixture was put back into the incubator for one hour to lift cells for counting and characterization.

### Example 13

Confocal microscopy. Cells were cultured on lattice matrices in the bioreactor for 7 days. Cells were washed using Ca⁺⁺ and Mg⁺⁺ PBS and fixed in place with 4% PFA for 15 minutes and washed again with PBS. Permeabilization was performed using 1% (W/V) Triton-X 100 in PBS for 30 minutes at 37° C. Cells were then washed and placed in 1% (W/V) Bovine Serum Albumin (BSA) and 0.1% (W/V) Triton-X 100 in PBS for one hour at room temperature. Cells were then stained for 30 minutes with 1 drop mL⁻¹ Phalloidin green (Invitrogen) and 1 μl mL⁻¹ DRAQ 5 resulting in a 5 mMol solution in the blocking solution. Cells were washed with Ca⁺⁺ and Mg⁺⁺ PBS and imaged on a Leica SP5 confocal microscope.

### Example 14

SEM imaging. PLA matrices were washed and prepared for electron microscopy. Samples were stuck to 0.5 in slotted stages (TED PELLA 16111) using conductive double-sided copper tape. Samples were imaged at 2 kV using Hitachi SU-70 scanning electron microscope.

### Example 15

Flow cytometry. hMSCs were cultured in experimental conditions and lifted with a 2:1 mixture of CDB and TrypLE-Express lifting cocktail to preserve cell surface markers. Cells were washed in PBS and placed in lifting cocktail for 15 minutes. After neutralization with fresh media, cells were fixed in 4% PFA for 15 minutes, washed twice with PBS, and blocked for 1 hour at room temperature. Blocking solution consisted of 1% (W/V) BSA and 0.1% (W/V) Triton-X 100 in PBS. Cells were stained for the positive markers CD105 (Invitrogen MHCD10520) and CD73 (Abcam ab157335) and were negative markers CD14 (Abcam ab91146) and CD19 (Abcam ab25510) at 1 μL per 500,000 cells in 500 μL following recommendations. Samples were then run at medium speed (35 μl/min) on a BD Accuri C6 flow cytometer and analyzed using FlowJo (Ashland, Oreg.). Unstained controls were used to gate cells. Fluorophore compensation was done through FlowJo and Fluorescence minus one (FMO) techniques.

### Example 16

hMSC Differentiation and staining. For both adipocyte and osteocyte differentiation, hMSCs were seeded at 12,000 cells cm² and cultured for 3 days in hMSC media following ATCC Toolkit protocols. ATCC differentiation toolkits for Osteocyte (PCS-500-052) and Adipocyte (PCS-500-050) differentiation were used. On the third day media was completely exchanged. For Adipocyte differentiation a conditioning pre-differentiation media was used, and every third day a ½ media change was performed with Adipocyte maintenance media. Osteocyte differentiation did not require a conditioning media and was maintained with only osteocyte toolkit media. On day 20, cells were washed with calcium magnesium free PBS and fixed by 4% PFA at room temperature for 15 minutes. Cells were washed and stained following respective protocols explained below. In brief, cells were washed with MQ water, and visualized on a phase contrast Olympus IX microscope.

### Example 17

Chondrocyte induction was performed according to a combination of ATCC protocols and previous research. Briefly, hMSCs were lifted from reactor using lifting cocktail, and counted. Cells were resuspended in chondrocyte differentiation toolkit (ATCC PCS-500-051) at 1.25×10⁶ cells mL⁻¹. 200 μl of cell laden media was put into 15 ml polypropylene falcon tubes and centrifuged at 270×g for 5 minutes and placed into incubator without resuspending cell pellet. When placed into incubator the tops of the tubes were loosened to allow gas exchange. After 24 hours the pellet was gently suspended via pipetting. Media was changed every 3 days for 21 days total. On day 21 cell aggregates were sliced into 8 μm thick samples using a HM 500 cryostat (Microm) and OTC compound (Tissue Tek 4583) and place onto glass slides. Samples were then stained and visualized on a phase contrast Olympus IX microscope.

Oil Red O was used to stain adipogenic differentiation of hMSCs. A working solution was prepared by mixing 3 ml of Oil Red solution (#0-1391, Sigma) 2 ml of MQ water immediately before. Cells were covered with oil red working solution and stained for 30 minutes at room temperature. Cells were washed twice with MQ water and visualized.

Alizarin Red stain was used to stain osteogenic differentiation of hMSCs. It arrived in working concentration at the proper pH, so no extra formulation was necessary. After fixation cells were washed twice with MQ water, then Alizarin red staining was overlaid onto the cells and left for 15 minutes. Cells were then washed three times with MQ Water and visualized.

Alcian blue was used to stain for chondrogenic differentiation of hMSCs. After cryostat slicing the samples were washed in Ca⁺⁺ Mg⁺⁺ PBS to preserve attachments while removing OTC compound, and fixed for 15 minutes in 4% PFA. The slides were then washed gently in DI water and alcian blue stain was overlaid onto the samples for 30 minutes. After 30 minutes the slides were rinsed with DI water, and then washed with 3% (V/V) glacial acetic acid solution in MQ water to remove excess dye. The cells were then gently rinsed again with DI water and visualized.

### Example 18

Computational fluid dynamic modeling. A simplified model was created in ANSYS 8.1 using a multiphase Volume of Fluid model in ANSYS FLUENT 18.2 (ANSYS Inc., Canonsburg, Pa.). The model consists of an inlet, the lattice made of crossing 0.4 mm square flow channels, a center void where the lattice would be anchored to its support in the system, and an outlet. Viscosity was modeled using Naiver-stokes equations and simulated using Standard K epsilon. Both energy and species transport were included. The SIMPLE pressure-velocity coupling scheme was used to run a transient model. Momentum convergence was set to 10⁻⁸. The inlet velocity was calculated by taking volumetric flow and dividing it by the diameter of the simulated inlet to give velocity. The model was validated comparing velocity in the model to dye experiments. Shear stress was calculated by Equation 3 using reported strain rate.

τ=ηγ Equation 3

Equation 3: Where τ is the shear stress, γ is the strain rate (s⁻¹), and η is the viscosity of the liquid. This was used in conjunction with the lowest flow rate needed to keep the lattice wetted.

### Example 19

Statistics. Graphs and statistics were done using Minitab 17 (Minitab Inc., PA). Error bars on graphs show 2 standard errors. Student's two-tailed t-test was used to determine significance for two data sets. Significance of multiple data sets was performed via one-way ANOVA and Tukey test.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification refers to at least one of something selected from the group consisting of A, B, C . . . and N, the text should be interpreted as requiring only one element from the group, not A plus N, or B plus N, etc.

What is claimed is:

1. A lattice structure for culturing cells, comprising:
a plurality of channels extending between a first channel pore surface and a second channel pore surface and each of the plurality of channels having a first channel pore and a second channel pore altogether forming a plurality of channel pores on each of the first channel pore surface and the second channel pore surface,
wherein each of the channel pores has an area of between about 0.01 mm² to about 1 mm², and
wherein the lattice structure is made of a biocompatible rigid material having a Young's modulus value of at least 0.5 GPa.

2. The lattice structure of claim 1, wherein the plurality of channels are approximately uniform with no more than 2% variation between any two of the plurality of channels.

3. The lattice structure of claim 1, wherein the first channel pore surface and the second channel pore surface have a diameter or diagonal of between about 5 mm to 100 mm.

4. The lattice structure of claim 1, wherein the lattice structure has a total space-occupying volume of between about 2 cm³ to about 800 cm³.

5. The lattice structure of claim 1, wherein each of the channel pores is approximately the same size having no more than 2% variation in any dimension, and each of the channel pores has a diameter or diagonal of between about 0.1 mm to 1 mm.

6. The lattice structure of claim 1, wherein each of the channel pores has approximately the same area size and the lattice structure further comprises a spacing distance between all of the channel pores that is equal to the area size of the channel pores.

7. The lattice structure of claim 1, wherein the biocompatible rigid material is selected from polylactic acid (PLA), polystyrene, polycarbonate (PC), polyethylene terephthalate glycol (PETG), thermoset polyurethane (TPU), polycaprolactone (PCL), or acrylonitrile butadiene (ABS).

8. The lattice structure of claim 1, wherein the first channel pore surface and the second channel pore surface have a perimeter edge forming a circular, oblong, square, octagonal, hexagonal, or rectangular shape.

9. The lattice structure of claim 1, wherein each of plurality of the channel pores have a square or circular shape.

10. The lattice structure of claim 1, further comprising a hollow passage through the lattice structure extending from the first channel pore surface to the second channel pore surface, the hollow passage allowing for insertion of a member for securing the lattice structure.

11. The lattice structure of claim 1, further comprising a removable substrate member made of the biocompatible rigid material and removably attached to at least one of the first channel pore surface or the second channel pore surface.

12. The lattice structure of claim 1, further comprising a surface coating, the surface coating selected from the group consisting of functional groups, denatured protein-based fibers, thermopolymers, plasma treatment, and/or sodium hydroxide.

13. The lattice structure of claim 12, wherein the thermopolymers are selected from poly(N-isopropylacrylamide) p(NIPAm), poly-(ethylpyrrolidone methacrylate) (pEPM), poly[2-(dimethylamino)ethyl methacrylate] (pDMAEMA), hydroxypropylcellulose, poly(vinylcaprolactame), or polyvinyl methyl ether.

14. The lattice structure of claim 12, wherein the denatured protein-based fibers are selected from gelatin, collagen type 1, collagen type 2, fibronectin, and/or laminin.

15. A system for culturing cells comprising:
a bioreactor assembly comprising the lattice structure of claim 1.

16. The system of claim 15, wherein the lattice structure further comprises a hollow passage extending from the first channel pore surface to the second channel pore surface, the hollow passage allowing for insertion of a member for securing the lattice structure within the bioreactor assembly.

17. The system of claim 15, wherein the bioreactor assembly is a perfusion bioreactor for cell expansion and recovery.

18. A method for maintaining cell type homogeneity or maintaining cell stemness throughout a culturing of a cell type, the method comprising:
culturing the cell type in a bioreactor assembly comprising the lattice structure of claim 1.

19. The method of claim 18, wherein the culturing comprises oxygen levels at between 1% to 20% oxygen (O₂).

20. A method for seeding cells by gravitational or perfusion flow, comprising culturing the cells using the system of claim 14.
